(19) United States
(12) Patent Application Publication
(10) Pub. No.: US 2008/0082090 A1
(43) Pub. Date: Apr. 3, 2008

(54) METHOD AND APPARATUS FOR DERMATOLOGICAL TREATMENT AND TISSUE RESHAPING

(75) Inventor: Dieter Manstein, Boston, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 11/952,492

(22) Filed: Dec. 7, 2007

Related U.S. Application Data
(63) Continuation-in-part of application No. 11/098,030, filed on Apr. 1, 2005.
(60) Provisional application No. 60/558,476, filed on Apr. 1, 2004.

(51) Int. Cl. A61B 18/20

(52) U.S. Cl. 606/9; 606/10; 607/89

(57) ABSTRACT

The present invention is directed to a method and apparatus for providing electromagnetic radiation or other energy to tissue. An array of needles can be inserted at least partially into the tissue, and energy, e.g., optical energy, can be provided to the needles. The needles can include an optical waveguide configured to direct the energy to needle tips located within the tissue adjacent to one or more target regions. The energy can thus be provided directly to the target regions through the needles without being absorbed by upper portions of the tissue. Such method and apparatus can be used to treat a variety of skin conditions, including wrinkles and pigmentation defects. One or more of the needles in the array can also be hollow and configured to provide an analgesic or other substance into the tissue near the target regions.

### CROSS-REFERENCE TO RELATED APPLICATIONS

This application is a continuation-in-part of U.S. patent application Ser. No. 11/098,030, filed on Apr. 1, 2005, and claims priority from U.S. Provisional Application Ser. No. 60/558,476, filed on Apr. 1, 2004, the entire disclosures of which are incorporated herein by reference in their entireties.

### FIELD OF THE INVENTION

The present invention is directed to a method and apparatus for skin treatment. More specifically, it is directed to the method and apparatus with which energy is applied to skin tissue using arrays of needles (or needlelike elements) to damage selected regions of the skin, and thereby promotes beneficial results, including skin tightening, tissue remodeling, and treatment of other skin disorders such as port wine stains and pigmentation defects.

### BACKGROUND INFORMATION

Skin is primarily made of two layers. The outer layer, or epidermis, has a thickness depth of approximately 100 μm. The inner layer, or dermis, has depth of approximately 3000 μm from the outer surface of the skin and is primarily composed of a network of protein fibers known as collagen, together with water. As provided herein, ‘dermal tissue’ can refer to both the dermis and the epidermis. The terms ‘dermal tissue’ and ‘skin’ can also be used interchangeably throughout the present disclosure.

There is an increasing demand for repair of skin defects, which can be induced by aging, sun exposure, dermatological diseases, heredity, traumatic effects, and the like. For example, aging skin tends to lose its elasticity, leading to increased formation of wrinkles and sagging. Other causes of undesirable wrinkles in skin include excessive weight loss and pregnancy.

There are several well-known surgical approaches to improving the appearance of skin by eliminating slackness that involve incisions being made in the skin and the removal of some tissue followed by rejoining of the remaining tissue. These surgical approaches include facelifts, brow lifts, breast lifts, and “tummy tucks.” Such approaches can produce a number of negative side effects including, e.g., scar formation, displacement of skin from its original location relative to the underlying bone structure, and uneven tightening.

Certain treatments which use electromagnetic radiation have been developed to improve skin defects by inducing a thermal injury to the skin, which results in a complex wound healing response of the skin and/or certain biological structures located therein, such as blood vessels. This can lead to a biological repair of the injured skin. Various techniques providing this effect have been introduced in recent years. These techniques can be generally categorized in two groups of treatment modalities: ablative laser skin resurfacing (“LSR”) and non-ablative collagen remodeling (“NCR”). The first group of treatment modalities, e.g., LSR, can cause fairly extensive thermal damage to the epidermis and/or dermis, while the second group, e.g., NCR, is designed to avoid thermal damage of the epidermis.

LSR is generally considered to be an effective laser treatment for repairing certain skin defects. In a typical LSR procedure, shown schematically in FIG. 1, a region of the epidermis 100 and a corresponding region of the dermis 110 beneath it are thermally damaged to promote wound healing. For example, electromagnetic energy 120 is directed towards a region of skin, thus ablating an upper portion of the skin and removing both epidermal and dermal tissue in region 130. LSR with pulsed CO₂ or Er:YAG lasers, which may be referred to in the art as laser resurfacing or ablative resurfacing, can be a treatment option for signs of photo-aged skin, chronically aged skin, scars, superficial pigmented lesions, stretch marks, and superficial skin lesions. However, certain patients may experience major drawbacks after such LSR treatment, including edema, oozing, and burning discomfort during first fourteen (14) days after treatment. These drawbacks can be unacceptable for many patients. LSR procedures can also be relatively painful and therefore generally may require an application of a significant amount of analgesia. While LSR of relatively small areas can be performed under local anesthesia provided by an injection of an anestheticum, LSR of relatively large areas can frequently be performed under general anesthesia or after nerve blockade by multiple injections of anesthetic.

A limitation of LSR is that this ablative resurfacing in areas other than the face generally may have a greater risk of scarring because the recovery from skin injury within these areas is not very effective. Further, LSR techniques are generally better suited for a correction of pigmentation defects and small lesions than for reducing or eliminating wrinkles.

In an attempt to overcome the problems associated with LSR procedures, several types of NCR techniques have emerged. These techniques are variously referred to in the art as non-ablative resurfacing, non-ablative subsurfacing, or non-ablative skin remodeling. NCR techniques generally utilize non-ablative lasers, flashlamps, ultrasound assisted devices, or radio frequency current to damage dermal tissue while sparing damage to the epidermal tissue. The concept behind NCR techniques is that thermal damage of the dermal tissue is thought to induce collagen shrinkage, leading to tightening of the skin above, and stimulation of wound healing which results in biological repair and formation of new dermal collagen. This type of wound healing can result in a decrease of structural damage related to photoaging. Avoidance of epidermal damage in NCR techniques can decrease the severity and duration of treatment-related side effects. In particular, post-procedural oozing, crusting, pigmentary changes and incidence of infections due to prolonged loss of the epidermal barrier function can usually be avoided by using NCR techniques.

In the NCR procedure for skin treatment, illustrated schematically in FIG. 2, selective portions of dermal tissue 135 within the dermal layer 110 are heated to induce wound healing without damaging the epidermis 100 above. A selective dermal damage that leaves the epidermis relatively undamaged can be achieved by cooling the surface of the skin and focusing electromagnetic energy 120, which may be a laser beam, onto a dermal region 135 using a lens 125. Other strategies can also be applied using nonablative lasers to achieve damage to the dermis while sparing the epidermis in NCR treatment methods. Nonablative lasers used in NCR procedures generally have a deeper dermal penetration depth as compared to ablative lasers used in LSR procedures. Wavelengths in the near infrared spectrum can be used. These wavelengths cause the non-ablative laser to have a deeper penetration depth than the very superficially-absorbed ablative Er:YAG and CO₂ lasers. Examples of NCR techniques and apparatus are described in U.S. Patent Publication No. 2002/0161357.

Although NCR techniques can assist in avoiding epidermal damage, they may have limited efficacies. An improvement of photoaged skin or scars after the treatment with NCR techniques can be significantly smaller than the improvements found when LSR ablative techniques are utilized. Even after multiple treatments, the clinical improvement is often below the patient's expectations. In addition, a clinical improvement may be delayed for several months after a series of treatment procedures. The NCR procedure can be moderately effective for wrinkle removal, and may generally be ineffective for dyschromia. One exemplary advantage of the NCR procedure is that it generally does not have the undesirable side effects that are characteristic of the LSR treatment, such as the risk of scarring or infection.

A further limitation of NCR procedures relates to the breadth of acceptable treatment parameters for safe and effective treatment of dermatological disorders. The NCR procedures generally rely on an optimum coordination of laser energy and cooling parameters, which can result in an unwanted temperature profile within the skin leading to either no therapeutic effect or scar formation due to the overheating of a relatively large volume of the tissue. In general, it may become more difficult to obtain a particular small and localized zone of thermal damage at increasing depth within the tissue.

Another approach to skin tightening and wrinkle removal involves the application of a radio frequency (“RF”) electrical current to the dermal tissue via a cooled electrode at the surface of the skin. An application of the RF current in this noninvasive manner can result in a heated region developed below the electrode that damages a relatively large volume of the dermis, and an epidermal damage is minimized by the active cooling of the surface electrode during treatment. This treatment approach can be painful, and may lead to a short-term swelling of the treated area. In addition, because of the relatively large volume of tissue treated and the need to balance application of the RF current with the surface cooling, this RF tissue remodeling approach may likely not allow a fine control of damage patterns and subsequent skin tightening. This type of RF technique is monopolar, and uses a remote electrical ground in contact with the patient to complete the current flow from the single electrode. The current in monopolar applications generally flows through the patient's body to the remote ground, which can lead to unwanted electrical stimulation of other parts of the body. In contrast, bipolar instruments can conduct current between two relatively nearby electrodes, and thereby through a more localized pathway.

Skin may also exhibit various discolorations or other pigmentation defects which may be aesthetically undesirable. Such defects can include, e.g., hemangiomas, port wine stains, varicose veins, rosacea, etc. Such skin disorders and discolorations may also be treated by application of light or other electromagnetic radiation (“EMR”) to the skin tissue. For example, port wine stains (“PWSs”) may be treated by applying electromagnetic radiation of certain wavelengths to the tissue containing the blood vessels, which make up the PWS. Such tissue may generally be located some distance below the outer surface of the skin tissue.

In general, application of EMR to skin or other tissue to treat such defects can be inefficient or lead to unwanted side effects. For example, FIG. 2 shows EMR 120 which is directed to a target area of tissue 135 which lies at some depth within the dermal skin tissue 110. Such energy 120 passes through a region of the epidermis 100 and an upper region of the dermis 110. A certain amount of the energy 120 may be absorbed and/or otherwise interact with this epidermal tissue 100 and/or dermal tissue 110 which lies above the target area 135, which can further lead to thermal damage or other unwanted interactions in the tissue which lies above the target tissue 135 being treated.

EMR having certain wavelengths may be highly absorbed in skin tissue, and can penetrate only a short distance below the surface before being substantially absorbed by the tissue. Thus, it may be difficult to provide such highly-absorbed EMR to a region of tissue which lies below the surface of the skin, and there may be significant undesirable absorption of such EMR in tissue which lies above the treatment region.

In view of the shortcomings of the above described procedures for dermatological treatment and tissue remodeling, it may be desirable to provide procedures and apparatus that can combine safe and effective treatment for tissue remodeling, skin tightening, wrinkle removal, and treatment of various skin conditions, discolorations, diseases and other defects. Such exemplary procedures and apparatus may preferably reduce or minimize undesirable side effects such as intra-procedural discomfort, post-procedural discomfort, lengthy healing time, heating or damage of healthy tissue, and post-procedural infection.

### SUMMARY OF THE INVENTION

It is therefore one of the objects of the present invention to provide exemplary apparatus and method that can combine safe and effective treatment for an improvement of dermatological disorders with minimum side effects. Another object of the present invention is to provide exemplary apparatus and method that promotes beneficial effects, e.g., skin tightening, wrinkle removal, and/or improvement of pigmentation defects, by creating a pattern of small localized regions of thermal damage within the dermis. Still another object of the present invention is to provide exemplary method and apparatus for skin tightening or other forms of tissue treatment by using an array of needles to controllably deliver electrical, thermal, optical and/or other electromagnetic energy to predetermined locations within the dermis or other tissue.

These and other objects can be achieved with an exemplary embodiment of the apparatus and method according to the present invention, in which portions of a target area of tissue are subjected to electromagnetic radiation, such as radio frequency pulses or optical energy. For example, an electromagnetic radiation can be directed to a target region within the skin or deeper tissue using minimally invasive method and apparatus, which can provide localized wounding or damage to the target area. Such wounding may be fractional, e.g., it can be provided to portions of the target region which are separated by undamaged or unwounded volumes of tissue. The electromagnetic radiation may be generated by an electromagnetic radiation source, which can be configured to deliver heat, radio frequency pulses, electrical current, optical energy, or the like to a plurality of target areas.

In yet another exemplary embodiment according to the present invention, an electromagnetic radiation source may be configured to generate electromagnetic radiation, and a delivery device comprising an array of needles, coupled to the electromagnetic radiation source, can be configured to penetrate the skin to one or more desired depths to deliver the electromagnetic radiation directly to a plurality of target areas in proximity to the tips of the needles.

Exemplary embodiments of the present invention can provide the method and apparatus in which an array of needles may be inserted into a region of skin, where the tips of the needles are configured to penetrate to one or more predetermined depths. Electromagnetic energy, e.g., optical energy, can then be provided through the needles to create regions of thermal damage and/or necrosis, or to achieve some further therapeutic effect, in the tissue surrounding the tips of the needles. The needles can be hollow and may contain a light guide or optical fiber. Alternatively, such needles may be formed by coating optical fibers or other waveguides with a rigid coating such as, e.g., a metallic coating or a diamond film. The needles may also include a rigid fiber or waveguide as a core, which may be coated with a material that can have reflective properties or a different refractive index than the core to help direct optical energy to the tip region of the needles. Optical energy or other EMR can be provided, e.g., by a laser, a flashlamp, etc.

In certain exemplary embodiments of the invention, one or more of the needles in the array can be hollow and may be used to deliver small amounts of analgesic or anesthetic into the region of skin being treated. Such exemplary hollow needles may be interspersed among the other needles in the array which are configured to deliver electromagnetic energy. Alternatively, such hollow needles may be configured as electrodes which can also deliver RF energy in addition to optical energy or analgesic or anesthetic.

In another exemplary embodiment of the present invention, certain needles in the needle array may also be connected to a second source of electrical current in the milliampere range. A detection of a nerve close to one or more inserted needles of the array can be performed by a sequential application of small currents to the needles in the array and observation of any visible motor response. Alternatively, other feedback techniques may be used to avoid thermal damage of a nerve fiber by a subsequent higher energy pulse such as, e.g., a direct feedback from the patient of a perceived sensation or an evaluation of evoked potentials triggered by such small current. If a nerve is detected, neighboring needle or needles can be deactivated during the subsequent application of RF current, optical energy, or other EMR to further needles in the array to avoid damaging the nerve.

In yet another exemplary embodiment of the invention, the methods and apparatus described herein can be used to heat portions of cartilage, such as that located in the nose, using a minimally invasive technique, which can allow reshaping of the pliant heated cartilage to a desired form.

These and other objects, features and advantages of the present invention will become apparent upon reading the following detailed description of embodiments of the invention, when taken in conjunction with the appended claims.

### BRIEF DESCRIPTION OF THE DRAWINGS

Further objects, features and advantages of the invention will become apparent from the following detailed description taken in conjunction with the accompanying figures showing illustrative embodiments, results and/or features of the exemplary embodiments of the present invention, in which:

FIG. 1 is a schematic diagram of a cross section of a tissue treated using a conventional ASR procedure;

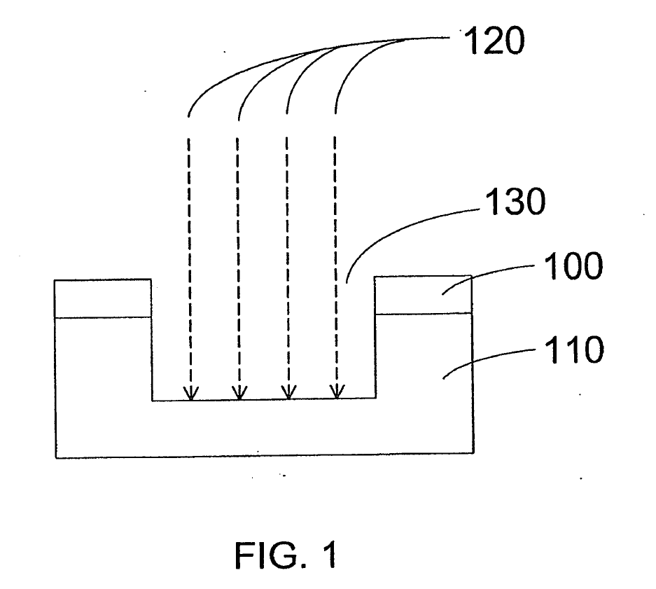

FIG. 2 is a schematic diagram of a cross section of a tissue treated using a conventional NSR procedure;

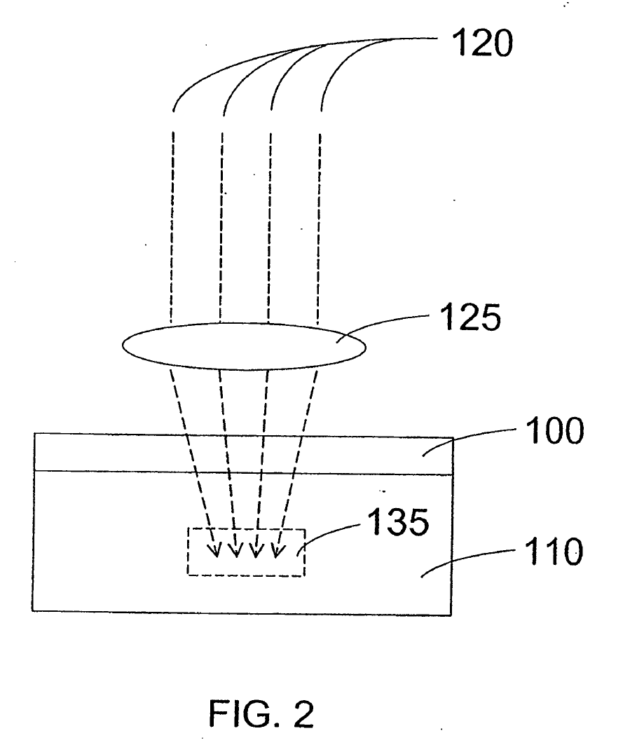

FIG. 3 is a schematic diagram of a cross section of a tissue treated using an exemplary apparatus and/or method in accordance with an embodiment of the present invention;

FIG. 4 is a schematic illustration of an apparatus for providing electromagnetic energy to tissue according to exemplary embodiments of the present invention; and

FIG. 5 is a schematic illustration of a further exemplary apparatus for providing electromagnetic energy to tissue according to exemplary embodiments of the present invention.

Throughout the drawings, the same reference numerals and characters, unless otherwise stated, are used to denote like features, elements, components, or portions of the illustrated embodiments. Moreover, while the present invention will now be described in detail with reference to the figures, it is done so in connection with the illustrative embodiments and is not limited by the particular embodiments illustrated in the figures.

### DETAILED DESCRIPTION OF EXEMPLARY EMBODIMENTS OF THE INVENTION

The present invention relates to exemplary methods and apparatus for improvement of skin defects, including but not limited to wrinkles, stretch marks, cellulite, discolorations, and other pigmentation defects. In one exemplary embodiment, skin tightening, tissue remodeling and/or pigmentation effects can be accomplished by creating a distribution of regions of necrosis, fibrosis, or other damage in a target region of the tissue. The tissue damage can be achieved by delivering localized concentrations of electrical current or electromagnetic radiation (e.g., light, laser, etc.) that can be absorbed by tissue and/or converted into heat in the vicinity of the tips of the needle electrodes. Inducing regions of local thermal damage within the dermis can, for example, result in an immediate shrinking of collagen, leading to beneficial skin tightening response. Additionally, the thermal damage can stimulate the formation of new collagen, which generally makes the local skin tissue fuller and gradually leads to additional skin tightening and reduction of wrinkles.

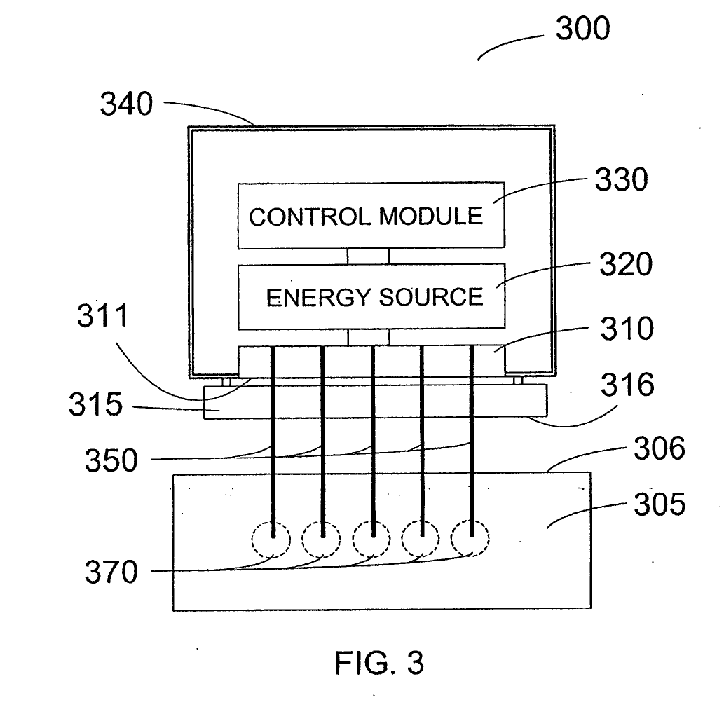

One exemplary embodiment of a tissue treatment apparatus 300 according to the present invention is shown in FIG. 3. This exemplary apparatus 300 can be used to create regions of damage within the tissue being treated. The exemplary apparatus 300 comprises a plurality of needles 350 attached to a base 310. The base is attached to housing 340 or formed as a part of the housing. A source of RF current 320 can be electrically connected to each of the needles 350. A control module 330 permits a variation of the characteristics of the RF electrical current, which can be supplied individually to one or more of the needles. Optionally, the current source 320 and/or the control module 330 may be located outside of the housing.

In one exemplary embodiment of the present invention, the current source 320 can be a radio frequency (RF) device capable of providing signals having frequencies in a desired range. In another exemplary embodiment, the current source 320 is capable of outputting an AC or DC electric current. The control module 330 may provide application-specific settings to the current source 320. The current source 320 can receive these settings, and generate a current directed to and from specified needles for selectable or predetermined durations, intensities, and sequences based on these settings.

In yet another exemplary embodiment of the present invention, a spacer substrate 315 containing a pattern of small holes through which the array of needles 350 protrudes may optionally be provided between the base 310 and the surface 306 of the skin 305. This spacer substrate 315 may be used to provide mechanical stability to the needles 350. Optionally, this substrate 315 may be movably attached to the base 310 or housing 340 and adjustable with respect to base 310. In this manner, the substrate 315 can be adjusted to specify one or more distances that the needles 350 protrude from the lower surface 316 of spacer substrate 315, thereby controlling or limiting the depth to which the needles 350 can be inserted into the skin 305.

In practicing an exemplary method in accordance with the present invention, the sharp distal ends of needles 350 can pierce the surface 306 of the skin tissue 305, and may be inserted into the tissue 305 until the bottom surface 316 of the spacer substrate 315 (or the bottom surface 311 of the base 310 if a spacer substrate 315 is not used) contacts the surface 306 of the skin 305. This configuration permits a reliable insertion of the array of needles to a predetermined depth within the tissue being treated. The control module 330 can be configured to deliver controlled amounts of RF current to one or more needles 350.

The base 310 and/or the spacer substrate 315, if provided, can be planar or may have a bottom surface that is contoured to follow the shape of the region of tissue being treated. For example, the bottom surface 311 of the base 310 can have a planar, convex, or concave contour. Such contour may be selected based on the area of skin being treated, e.g., to more closely conform to the shape of the skin surface above the region of tissue being treated. This exemplary configuration can allow, for example, the penetration of the needles in the needle array to a uniform depth within the targeted tissue even if the surface of the skin is not planar, e.g., along the eye sockets, on a chin or cheek, etc. It may generally be preferable to provide needles that are substantially parallel in the needle array to allow for an easier insertion of the needle array into the skin.

In another exemplary embodiment of the present invention, the base 310 and/or the spacer substrate 315, if used, may be cooled using any suitable technique (for example, embedded conduits containing circulating coolant or a Peltier device). Such cooled base 310 or substrate 315 can thereby cool the surface 306 of the skin 305 when the needle array 350 penetrates the skin to reduce or eliminate pain. The surface region of the skin being treated and/or the needles 350 may also be precooled, e.g., using convective or conductive techniques, prior to penetration of the skin by the array of needles 350.

In a further exemplary embodiment of the present invention, the shafts of needles 350 can be conductive and electrically insulated except for a portion of the needle near the tip and/or one or more locations along the length of the needle 350. In the exemplary apparatus shown in FIG. 3, application of the RF current to the needles 350 can generate heat near the uninsulated tip, which can further generate thermal damage in regions 370 around the tip of each needle. If certain portions along the needles 350 are also not insulated, thermal damage may also be generated around these non-insulated portions. The thermally damaged regions 370 can be obtained from operation of the exemplary apparatus 300 in, e.g., a monopolar configuration, in which a remote grounding electrode (not shown in FIG. 3) can be attached to a remote part of the patient's body to complete the circuit of electricity conveyed to the needles 350 by the energy source 320. In this exemplary monopolar configuration, the RF current can generate heating around the tip regions of the needles 350, thus generating thermal damage in the tissue regions 370 adjacent to the needle tips which may be, e.g., approximately spherical or slightly elongated in shape.

In a further exemplary embodiment of the present invention, the current may be delivered simultaneously to all needles 350 in the needle array to produce a pattern of thermal damage around the tip of each of the needles 350. In alternative exemplary embodiments, the control module 330 and/or the energy source 320 can be configured to supply electrical current to individual needles 350, to specific groups of such needles 350 within the array, or to any combination of the individual needles 350 in a variety of specified temporal sequences. For example, providing the current to different needles 350 at different times during treatment (e.g., instead of providing current to all needles 350 in the array at once) may help to avoid potential local electrical or thermal interactions among the needles 350 which can lead to an excessive local damage.

In yet another exemplary embodiment of the present invention, one or more vibrating arrangements, such as a piezoelectric transducer or a small motor with an eccentric weight fixed to the shaft, may be mechanically coupled to the housing 340 and/or the base 310 that generally supports the array of needles 350. The vibrations conductively induced in the needles 350 by such vibrating arrangement can facilitate a piercing of the skin surface 306 by the needle tips and subsequent insertion of the needles 350 into the tissue 305. The vibrating arrangement can have an amplitude of vibration in the range of about 50-500 μm, and preferably between about 100-200 μm. The frequency of the induced vibrations can be between about 10 hz and about 10 khz and preferably between about 500 hz and about 2 khz, and more preferably about 1 khz. The particular vibration parameters chosen may depend on the size and material of the needles, the number of needles in the array, and the average spacing, or lateral distance, between the needles. The vibrating arrangement may further include an optional controller configured to adjusting the amplitude and/or frequency of the vibrations.

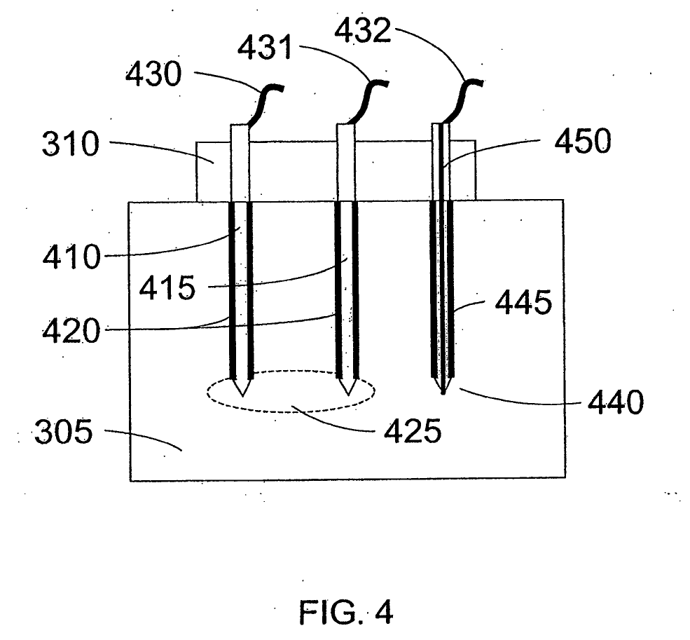

Further details of the exemplary embodiments of the present invention are shown in FIG. 4. For example, conductive needles 410, 415 are shown attached to the base 310. An insulation 420 covers a shaft of needles 410, 415 protruding from the base 310 except for a portion near the lower tip, and can electrically insulate each conductive needle shaft from the surrounding tissue 305. Electrical conductors 430, 431, which may be wires or the like, extend from an upper portion of the needles 410, 415, respectively, and are connected to the energy source (not shown in FIG. 4). Suitable insulating materials for the insulation 420 can include, but are not limited to, Teflon®, polymers, glasses, and other nonconductive coatings. Insulator materials may be chosen, e.g., to facilitate penetration and insertion of the needles 410, 415 into the tissue 305.

The needles 410, 415 can operate in a bipolar mode according to another exemplary embodiment of the present invention. For example, the needle 410 can be a positive electrode delivering RF or other current to the tip portion of the needle from the energy source via a conductor 430. The needle 415 can be a grounding electrode that is connected to a ground potential of the energy source via a conductor 431. In this exemplary configuration, the applied current can travel through the skin tissue 305 between the tips of the needles 410, 415, thus generating an elongated region of a thermal damage 425. Such bipolar operation can be used to generate a number of such elongated regions of damage 425, which can be located around and/or between the tips of adjacent or nearby needles 410, 415 in the needle array.

An elongated region of the damaged tissue 425 can be generated between two adjacent or nearby needles 410, 415 in the needle array using a bipolar mode through an appropriate configuration of the control module 330 and the energy source 320. For example, the elongated damage regions 425 can be formed between several pairs of the needles 410, 415 within the array of needles to form a desired damage pattern in the tissue 305. The regions of the thermal damage 325, which may be created using the exemplary needle array apparatus in a bipolar mode, can be formed simultaneously or, alternatively, sequentially, using any combinations of proximate needles in the array to form each region. A variety of thermal damage patterns can be created using a single array of the needles 410, 415 through appropriate configuration of the energy source 320 and the control module 330 to deliver predetermined amounts of current between the selected pairs of the needles 410, 415. The exemplary apparatus thus can generate complex damage patterns within the tissue 305. Such damage patterns may be configured, e.g., to be macroscopically elongated in a particular direction to produce anisotropic shrinkage and reshaping, or to approximately match a shape of a pigmentation defect, etc.

In an exemplary embodiment of the present invention, the array of needles may include pairs of needles which can be provided relatively close to each other and separated from adjacent pairs by larger distances. Such exemplary geometry may be preferable for generating damage in a bipolar mode between such pairs of needles. Needles may also be arranged in a regular or near-regular square or triangular array. In any such array geometry, the pattern of damage and resultant tissue reshaping may be controlled with some precision by adjusting the intensity and duration of power transmitted to single needles and/or to certain pairs of needles.

The amount of energy directed to a given needle can be selected or controlled based on the tissue being treated and the desired amount of thermal damage to be provided. For exemplary needle spacings described herein, the energy source can be configured to deliver about 1-100 mJ per needle or pair of needles in the array. It may be preferable to initially use lower amounts of energy, and perform two or more treatments over a particular target area to better control the damage patterns and extent of reshaping.

In exemplary embodiments of the present invention, certain ones of the needles can have a width of less than about 1000 μm, or less than about 800 μm. Needles having less than about 500 μm in diameter may also be used if they are mechanically stiff for reliable insertion into skin tissue. For example, such thinner needles can be formed buy coating optical fibers or the like with a rigid coating such as, e.g., a metallic layer or a diamondlike carbon film. Needles thicker than about 1000 μm in diameter may also be used in accordance with certain exemplary embodiments of the invention, but such larger needles may be undesirable because of the difficulty in forcing larger needles to penetrate the skin, and because of an increased likelihood of pain and/or scarring when using larger needles.

A length of the needles extending into the skin (e.g., the lengths of the needles 410, 415, 440 which protrude from a lower face of the base 310 as shown in FIG. 4) can be selected based on a targeted depth for damaging the tissue. An exemplary depth for targeting collagen in the dermis can be about 1500-2000 μm, although shallower or deeper distances may be preferred for different treatments and regions of the body being treated. For example, needle lengths may be selected for a particular treatment to correspond to an approximate depth below the skin surface of a particular defect (e.g., a port wine stain, a hemangioma, etc.).

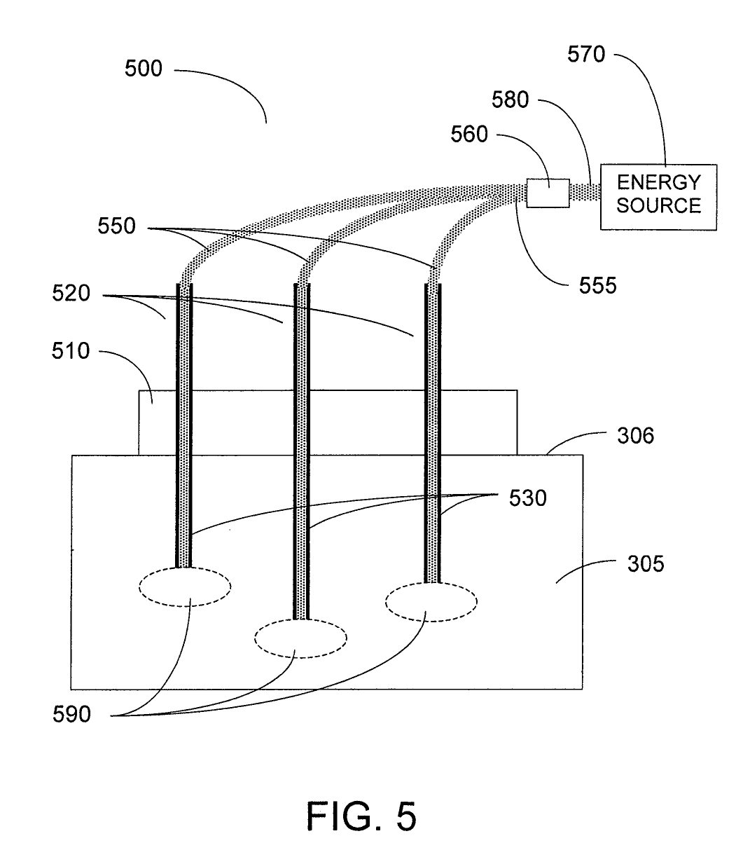

In certain exemplary embodiments of the present invention, the needles within a single array may have different lengths (e.g., they may extend by different lengths from the base 310 or the spacer substrate 315 shown in FIG. 3). An exemplary needle length variation which may facilitate the positioning of tips of needles 520 at different depths within the tissue being treated is shown, e.g., in FIG. 5. Such length variation of the needles 520 in a needle array can generate, e.g., thermal damage of tissue at more than one depth or over a range of depths within the skin based on a single insertion of the needle array into skin tissue. This variation in needle lengths (and corresponding variation in insertion depths) can be used, for example, to generate a larger volume of heated and/or damaged tissue below the skin surface, which can be used to treat larger defects in the skin and/or produce a more pronounced shrinkage response.

The exemplary needle arrays may have any geometry appropriate for the desired treatment being performed. The spacing (e.g., lateral distance) between the adjacent needles may be less than about 1 cm, or preferably less than about 8 mm. Optionally, the spacing between the adjacent needles in the array may be less than about 5 mm, or less than about 2 mm. The spacing between the needles in the array does not have to be uniform, and can be smaller in areas where a relatively greater amount of damage or more precise control of the damage in the target area of the tissue is desired. Various numbers of needles may be used in exemplary needle arrays. For example, the needle arrays in accordance with the exemplary embodiments of the present invention may include at least about 10 needles, at least about 30 needles, or at least about 50 needles. Arrays having a larger number of the needles can be used, e.g., to treat a larger volume of tissue with a single insertion of the needle array into the skin, and/or to provide energy to more closely-spaced target areas within the tissue.

In yet another embodiment of the present invention, one or more of the needles in the array may be hollow, such as the needle 440 shown in FIG. 4. The center channel 450 may be used to deliver a local analgesic such as, e.g., lidocaine 2% solution from a source (not shown) located within or above the base 310 into the tissue 305 to reduce or eliminate pain caused by the thermal damage process.

In yet another exemplary embodiment of the present invention, one or more hollow needles 440 can be bifunctional, e.g., configured to conduct the RF current or other energy via the conductor 432, and also to deliver a local analgesic or the like through the center channel 450. The bifunctional needle 440 can also have an insulation 445 covering or extending around at least a portion of the shaft extending from base 310, e.g., except for the region near the lower tip. Analgesic may be supplied to the tissue either before or during application of the RF or other current to the needle 450.

In one exemplary embodiment of the present invention, one or more of the needles in the array may be bifunctional as described herein, such as the needle 440. Alternatively, one or more of the needles may be hollow and optionally nonconductive, and configured only to deliver a local analgesic or the like. The array of needles used for a particular treatment may include, for example, any combination of solid electrodes, bifunctional needles, or hollow nonconductive needles. For example, an exemplary needle array may include pairs of electrode needles operating in bipolar mode, with one or more hollow needles provided between or in proximity to each such pair. In this exemplary configuration, the hollow needles can deliver the analgesic to the tissue between or close to the tips of the electrode needles prior to applying current to the electrodes. Thus, a pain sensation can be reduced or eliminated in the tissue that is thermally damaged by the electrode needles.

In yet another exemplary embodiment of the present invention, one or more needles in the array may be connected to an electronic detection apparatus, and may be configured to detect a presence of a nerve near a needle tip. The electronic detection apparatus may include a source of electrical current in the milliampere range, and a control arrangement configured to transmit small currents (e.g., on the order of one or a few milliamps) to particular needles in the array. A detection of a nerve near any of the inserted needles of the array can be performed by sequential application of such small currents to the needles in the array, followed by observation of any visible motor response which can indicate presence of a nerve in proximity to a particular needle provided with such small current. If a nerve is detected, the control module 330 can be configured to deactivate the needle or needles close to the detected nerve during the subsequent treatment to avoid damaging the nerve. A nerve detection technique based on similar principles is described, e.g., by Urmey et al. in Regional Anesthesia and Pain Medicine 27:3 (May-June) 2002, pp. 261-267.

In further exemplary embodiments of the present invention, an optical energy may be provided to target regions of tissue below the skin surface using the exemplary needle arrays as described herein. An exemplary apparatus 500 for providing the optical energy to the tissue in accordance with exemplary embodiments of the present invention is shown in FIG. 5. Such apparatus 500 can include a plurality of optical needles 520, which may be affixed to a substrate 510. An exemplary optical needle 520 can include an optical guide 550 provided in a rigid shell 530. The shell can have a form, e.g., of a hollow needle formed of metal or some other structurally rigid material. The optical guide 550 can be, e.g., an optical fiber or a waveguide configured to propagate optical energy to a distal end of the optical guide 550.

A distal end of the optical guide 550 can be provided near a tip of the optical needle 520, for example, in proximity to a distal end of the shell 530 such that, e.g., the end of the optical guide 550 may be located within the end of the shell 530, it can be provided approximately flush with the distal end of the shell 530, or it may alternatively protrude slightly beyond the end of the shell 530. Each optical needle 520 can thereby be configured to direct the optical energy through its length and into a target region of tissue 590 near the needle tip. For example, such optical needles 520 can direct the optical energy to such target regions 590 below the skin surface, where the optical energy is provided through at least a portion of the optical needle 520 and thereby may not be absorbed by the tissue located above the target regions 590.

In still further exemplary embodiments of the present invention, the optical guide 550 can be provided as part of a bundle 555 of such guides such as, e.g., an optical fiber bundle. An end of the bundle 555 can be affixed to a coupler 560 such as, e.g., an optical coupler. The coupler 560 can be further provided in communication with an energy source 570 using, e.g., a waveguide 580. Such exemplary apparatus 500 can facilitate connection and separation of an optical needle arrangement from the energy source 570, where the optical needle arrangement can include the fiber bundle 555, together with needles 520, substrate 510, and optical guides 550.

The exemplary optical needles 520, or any other needles used in a needle array as described herein, may be provided with different lengths as shown in FIG. 5. Such variation of needle lengths can provide optical energy or other forms of energy at a plurality of target regions 590 located at different depths within the skin tissue. Alternatively, the needles 520 in an exemplary needle array can be provided with a single length to direct energy to the target regions 590 located at a particular depth.

An exemplary optical needle 520 may be provided in a variety of forms. For example, such optical needle 520 can include an optical guide 550 provided in a rigid shell 530, such as a hollow needle, as described herein. This exemplary needle 520 can also be provided, e.g., as a shell 530 which may be deposited or coated on a portion of the optical guide 550. For example, an exemplary shell 530 can be formed of a metal or alloy, a ceramic, diamond or a diamondlike coating, etc. The shell 530 can be provided on the optical guide 550 using one or more deposition or coating techniques including, e.g., chemical-phase vapor deposition, physical vapor deposition, dip-coating of a solution, a sol-gel reaction, etc. If the optical guide 550 is coated with a shell 530 as described herein and the distal end of such optical guide 550 is covered with the coated material, the distal end can be, e.g., cut or abraded to expose the distal end of the optical guide 550. The distal end can be cut or abraded to form, e.g., a sharp point or another shape which can facilitate penetration of the distal end of such optical needle 520 thus formed into skin or other tissue.

In still further exemplary embodiments of the present invention, the energy source 570 can be selected based on the treatment to be performed. For example, the energy source 270 may include, but is not limited to, a diode laser, a diode-pumped solid state laser, an Er:YAG laser, a Nd:YAG laser, an argon-ion laser, a He—Ne laser, a carbon dioxide laser, an excimer laser, a pulsed dye laser, an intense pulsed light source, a flashlamp, or a ruby laser. Energy provided to the target areas of the tissue using the exemplary needle arrays may optionally be continuous or pulsed, with pulse and/or exposure durations selected based on the treatment being performed.

For example, pigment discolorations such as, e.g., port wine stains or hemangiomas can be treated by applying optical energy that may be strongly absorbed by hemoglobin in accordance with exemplary embodiments of the present invention. An optical needle array such as the exemplary array 500 shown in FIG. 5 can be used to provide such optical energy, e.g., blue light having a wavelength, directly to target regions below the skin surface containing the pigmentation defects. The applied energy may thus be provided directly to a plurality of target regions, and may not be absorbed by tissue located above such target regions.

Such exemplary delivery technique as described herein may be particular suitable for delivering electromagnetic radiation having a wavelength that is strongly absorbed by chromophores within the skin, and therefore may not otherwise penetrate to deeper regions of the skin tissue. For example, treatment-resistant port wine stains may particularly benefit from such exemplary delivery techniques in accordance with exemplary embodiments of the present invention. A limited efficacy of conventional delivery techniques for radiation having such selectively absorbed wavelengths (produced, e.g., by a pulse dye laser, an Alexandrite laser, a KTP laser, etc.) can result from insufficient penetration of such radiation to tissue locations that lie within deeper regions of the dermis.

Exemplary embodiments of the present invention may also provide delivery of radiation having strong hemoglobin-absorbed wavelengths, e.g. between about 380 nm and about 480 nm, into skin tissue. Delivery of ultraviolet (“UV”) radiation to skin tissue below the epidermis with minimal or no absorption by, or interference with, the epidermis can also be provided as described herein. Such delivery method and apparatus may provide particular benefits for therapies which include UV-activated drugs or other conditions that may effectively be treated with UV therapy such as, e.g., psoriasis, where conventional UV-based therapies may cause unwanted long-term side effects in the epidermis including, e.g., skin cancer.

Exemplary embodiments of the present invention can also be used for a broad range of treatment techniques in which the optical energy or other electromagnetic radiation may be applied to certain regions of skin tissue or other types of tissue. An effective treatment of such tissue, including treatment of various skin conditions, can be achieved using smaller amounts of applied energy (e.g., lower fluence or intensity, and/or fewer or shorter pulses) as compared to conventional treatments in which energy is directed onto the skin surface and then travels through an upper portion of the tissue to the target region. The energy provided by the energy source 570 can be directed to the target regions 590 near the tips of optical needles 520 with small loss of such energy in the optical guides 550, and little or no absorption of such energy by tissue lying above the target regions 590. Appropriate amounts of energy which can be applied using the exemplary optical needle arrays as described herein can be selected, for example, based on the amount of energy which can be estimated to reach the target regions in conventional treatments after a portion of such energy directed into the skin can be absorbed by the tissue located above the target regions. Thus, the exemplary embodiments of the present invention can provide effective treatment of skin conditions using less energy than that used in the conventional treatment techniques. Both safety and efficacy of such treatments can be improved through an application of the optical energy directly to the desired target regions using the exemplary optical needle arrays as described herein.

The exemplary embodiments of the present invention can be particularly beneficial for treating skin having dark pigmentation. For example, such darkly pigmented skin may tend to strongly absorb optical energy, such that most of such optical energy may be absorbed close to the skin surface, e.g., before a sufficient amount can penetrate to the depth of the target regions 590. Exemplary optical needle arrangements as described herein can facilitate such energy to “bypass” upper regions of skin tissue near the surface, and be applied directly to the target regions 590 at one or more particular depths within the skin.

Certain exemplary embodiments of the present invention can be used, for example, in photodynamic therapy (“PDT”) procedures. Conventional PDT techniques can include involves a local or systemic application of a light-absorbing photosensitive agent, or photosensitizer, which may accumulate selectively in certain target tissues. Upon an irradiation with the electromagnetic radiation, such as visible light of an appropriate wavelength, reactive oxygen species (e.g., singlet oxygen and/or free radicals) may be produced in cells or other tissue containing the photosensitizer, which can promote cell damage or death. The oxidative damage from these reactive intermediates can generally be localized to the cells or structures at which the photosensitizer is present. PDT treatments may therefore be capable of ‘targeting’ specific cells and lesions, for example, if the photosensitizer is present in significant quantity only at desired target sites and/or light activation is performed only at such target sites. Exemplary optical needle arrays in accordance with the exemplary embodiments of the present invention can be used to direct optical energy to particular target regions containing the photosensitizer. Thus, more effective PDT treatments can be achieved, including PDT treatment of skin having a dark pigmentation which may preclude a sufficient penetration of the optical energy to target regions within the skin when using conventional PDT techniques. Also, the exemplary delivery method and apparatus described herein may help to reduce or prevent certain undesirable side effects associated with conventional PDT techniques, including pain and/or induction of increased pigmentation.

Certain treatments performed in accordance with exemplary embodiments of the present invention may be used to target collagen in the dermis. This can lead to an immediate tightening of the skin, and a reduction of wrinkles overlying the damaged tissue which may be caused by contraction of the heated collagen. Over time, such thermal damage can also promote a formation of new collagen, which may further smooth an appearance of the skin.

Certain treatments performed in accordance with the present invention may be used to target collagen in the dermis. This can lead to immediate tightening of the skin and reduction of wrinkles overlying the damaged tissue arising from contraction of the heated collagen. Over time, the thermal damage also promotes the formation of new collagen, which serves to smooth out the skin even more.

Exemplary embodiments of the present invention may also be used to reduce or eliminate the appearance of cellulite. To achieve this, the exemplary arrays of needles can be configured to target the dermis and optionally the upper layer of subcutaneous fat directly. Creating dispersed patterns of small thermally-damaged regions in these layers can tighten the networked collagen structure, and likely suppress the protrusion of the subcutaneous fat into the dermal tissue that can cause cellulite.

Further exemplary methods and apparatus in accordance with the present invention can be used to reshape cartilage. For example, heating the cartilage to about 70 degrees C. can soften the cartilage sufficiently to permit reshaping that may persist after subsequent cooling. Currently, specialized lasers may be used to heat and soften cartilage in the nasal passages for reshaping. Using the methods and apparatus described herein, the cartilage can be targeted by an array of needles and heated in a suitably gradual way, using lower power densities and longer times, to provide relatively uniform heating. Shaping of the cartilage is thus possible using a minimally invasive technique that can be used where laser heating may not be feasible.

Any of the thermal damaging and tissue reshaping methods practiced in accordance with the present invention may be performed in a single treatment, or by multiple treatments performed either consecutively during one session or at longer intervals over multiple sessions. Individual or multiple treatments of a given region of tissue can be used to achieve the appropriate thermal damage and desired cosmetic effects.

The foregoing merely illustrates the principles of the invention. Various modifications and alterations to the described embodiments will be apparent to those skilled in the art in view of the teachings herein. It will thus be appreciated that those skilled in the art will be able to devise numerous techniques which, although not explicitly described herein, embody the principles of the invention and are thus within the spirit and scope of the invention.

What is claimed:

1. An apparatus for providing an electromagnetic radiation, comprising:
a plurality of needles, wherein at least two of the needles are configured to perforate a surface of a skin to at least one predetermined depth, and wherein each of the at least two of the needles are configured to direct the electromagnetic radiation to at least one predetermined target region located below the skin surface after the at least two needles have perforated the surface.

2. The apparatus of claim 1, further comprising a substrate configured to provide a first needle of the at least two needles in a particular location relative to and away from a placement of a second needle of the at least two needles.

3. The apparatus of claim 1, wherein the at least one predetermined target region is located in proximity to a distal end of at least one needle of the at least two needles.

4. The apparatus of claim 2, wherein each of the at least two needles comprises an optical guide.

5. The apparatus of claim 4, wherein the optical guide is at least one of a waveguide or at least a portion of an optical fiber.

6. The apparatus of claim 4, wherein a lateral distance between the first needle and the second needle is less than about 1 cm.

7. The apparatus of claim 4, wherein a lateral distance between the first needle and the second needle is less than about 8 mm.

8. The apparatus of claim 4, wherein a lateral distance between the first needle and the second needle is less than about 5 mm.

9. The apparatus of claim 4, wherein a lateral distance between the first needle and the second needle is less than about 2 mm.

10. The apparatus of claim 4, wherein a diameter of at least one of the at least two needles is less than about 1000 μm.

11. The apparatus of claim 4, wherein a diameter of at least one of the at least two needles is less than about 800 μm.

12. The apparatus of claim 4, wherein a diameter of at least one of the at least two needles is less than about 500 μm.

13. The apparatus of claim 4, wherein the plurality of needles includes at least about 10 needles.

14. The apparatus of claim 4, wherein the plurality of needles includes at least about 30 needles.

15. The apparatus of claim 4, wherein the plurality of needles includes at least about 50 needles.

16. The apparatus of claim 3, wherein the electromagnetic radiation is provided by at least one of a diode laser, a diode-pumped solid state laser, an Er:YAG laser, a Nd:YAG laser, an argon-ion laser, a He—Ne laser, a carbon dioxide laser, an excimer laser, a pulsed dye laser, a KTP laser, a fiber laser, an LED, an intense pulsed light source, a flashlamp, or a ruby laser.

17. The apparatus of claim 16, wherein the electromagnetic radiation is provided as a plurality of pulses.

18. The apparatus of claim 1, wherein the at least one predetermined depth includes a plurality of predetermined depths.

19. The apparatus of claim 4, further comprising a coupling arrangement configured to provide an optical guide in communication with a source of the electromagnetic radiation.

20. The apparatus of claim 4, further comprising at least one further needle which is hollow and configured to provide at least one of an analgesic, an anaesthetic, or a biologically active material to at least one further target region located below the skin surface.

21. The apparatus of claim 4, further comprising at least one radio frequency needle which is configured to provide a radio frequency electromagnetic energy to at least one additional target region located below the skin surface.

22. A method for applying an electromagnetic radiation, comprising:
controlling a source arrangement to generate the electromagnetic radiation; and
providing the electromagnetic radiation to at least two needles of a plurality of needles provided at least partially within the tissue,
wherein the at least two needles of the needles are configured to direct the electromagnetic radiation to a distal end of the at least two needles,
wherein, after the at least two needles perforate a surface of the tissue and distal ends thereof reach at least one predetermined location, the electromagnetic radiation travels through at least a portion of the at least two needles, and
wherein at least a portion of the electromagnetic radiation is provided to a region of tissue located in proximity to the distal end at or near the at least one predetermined location.

23. The method of claim 22, wherein each needle of the at least two needles comprises an optical guide.

24. The method of claim 22, wherein the electromagnetic radiation source is at least one of a diode laser, a diode-pumped solid state laser, an Er:YAG laser, a Nd:YAG laser, an argon-ion laser, a He—Ne laser, a carbon dioxide laser, an excimer laser, a pulsed dye laser, a KTP laser, a fiber laser, an LED, an intense pulsed light source, a flashlamp, or a ruby laser.
